(19) United States
(12) Patent Application Publication
(10) Pub. No.: US 2021/0007972 A1
(43) Pub. Date: Jan. 14, 2021

(54) Topical treatment for warts using film forming polymers

(71) Applicant: Pjetur Kristjan Nordal, Calgary (CA)

(72) Inventor: Pjetur Kristjan Nordal, Calgary (CA)

(21) Appl. No.: 17/032,519

(22) Filed: Sep. 25, 2020

Related U.S. Application Data
(63) Continuation of application No. 15/715,118, filed on Sep. 25, 2017, now abandoned.

(51) Int. Cl. A61K 9/00; A61K 31/717; A61K 33/00; A61K 31/78; A61K 31/19; A61K 31/80; A61K 9/70

(52) U.S. Cl. CPC A61K 9/0014; A61K 31/717; A61K 33/00; A61K 47/38; A61K 31/19; A61K 31/80; A61K 9/7015; A61K 31/78

(57) ABSTRACT

Human warts have been the subject of many treatment options. Most of the effective strategies involve invasive procedures or painful treatments. The goal of this treatment is to find a topical application of a compound that will remove the infected tissue without significant discomfort to the patient. The present invention effects the treatment by using a polymerizing compound that forms a boundary between the hyperkeratotic layer of infected epithelial cells and the healthy skin cells. This modality is both painless and does not harm healthy cells.

### CROSS-REFERENCE TO RELATED APPLICATIONS

This application is a continuation of U.S. patent application Ser. No. 15/715,118, filed on Sep. 25, 2017, the entirety of which is incorporated herein by reference.

### BACKGROUND

Warts or verrucae vulgaris, are common and cause pain and discomfort. Warts impact from 6 to 10% of the population and are generally considered benign. Warts are benign growths of the skin caused by a virus that involves the epidermis. Though warts are common their epidemiology is not well understood. There are over 60 serotypes of the Human papillomavirus (HPV) which are the causative agent of warts. The wide variety of viruses that cause warts makes the treatment complex.

Once developed, warts can be spread to other parts of the body or to other persons through skin-to-skin contact or contact with a surface contaminated with HPV. Warts are more commonly diagnosed based on their physical appearances and locations on the body. Generally, five different types of warts are classified by their clinical presentation.

Common Warts
(verrucae vulgaris) are domed and display hyperkeratosis. These warts may occur anywhere on the body, but typically not including genital regions, mucous membranes or plantar surfaces (soles or bottoms of the feet).

Periungual Warts
Occur on the skin surrounding finger and toe nails.

Flat Warts
(verrucae planae) usually occur on the face, trunk and extremities. They often occur on the faces and extremities of children and on the lower legs of women.

Plantar Warts
(verrucae plantares) occur only on the soles of the feet. Such warts typically become callused and grow into the foot due to the forces exerted on the foot from everyday movement.

Genital Warts
(Condylomata acuminata) are venereal warts that occur on the genitals and mucous membranes

Current Treatment Modalities
Many treatments of verrucae involve physical destruction of the infected cells. Choice of treatment depends on the location, size, number, type of wart, age and cooperation of the patient. The current treatments cause pain and or the destruction of healthy epidermal cells.
The treatment modalities include: chemical destruction, cryosurgery, electrodessication, laser surgery, and immunotherapy.

Chemical Destruction
The chemical destruction of cells is intended to peel off infected skin using caustics and acids. Examples of these chemicals are salicylic acid, lactic acid and trichloroacetic acid. Salicylic acid in a topical composition is available for the treatment of warts. In this form, Salicylic acid is a keratolytic agent that softens the hyperkeratotic areas by dissolving the intercellular matrix and enhancing shedding of scales.
Cantharidin is another form of chemical destruction of skin cells. It is an extract of the green blister beetle that leads to blistering and focal destruction of the epidermis. These acids do not bind preferentially to the wart-infected cells and can cause destruction and produce scarring of the surrounding normal skin if not carefully applied.
Podophyllin resin is also a chemotherapeutic agent employed for venereal warts which is more effective on mucosal surfaces.

Cryotherapy
The two most commonly used agents are carbon dioxide snow and liquid nitrogen.
Cryotherapy works mainly by destroying the host cell and stimulating an immune response to the area.
Freezing time can be as short as 5 seconds with liquid nitrogen and well over a minute with carbon dioxide snow. Usually, two freeze-thaw cycles are performed. The patient experiences pain and burning during the cryotherapy. After the wart thaws, the patient may experience a persistent throbbing pain that usually subsides by the end of the day.

Electrodessication and Curettage
It physically removes the HPV infected epidermal cells by scraping down to the dermis. The scraping is then paused while an electrosurgical device like a hyfrecator is used. Electrocoagulation (electrodessication) is then performed over the raw surgical ulcer to denature a layer of the dermis

Lasers
Laser treatment modalities are often painful and may require anesthesia and cause scarring.

Immunotherapy
A new immunomodulator, Imiquimod has recently been used to topically treat genital and perianal warts.

All these known treatments have a variety of disadvantages. Such disadvantages include ineffectiveness (e.g. chemical treatments and cryotherapy), undesirable pain and prolonged recovery (e.g. cryotherapy and lasers), undesirable scarring (e.g. surgical excision), and high expense (e.g. bleomycin injections to cause acute tissue necrosis).

### SUMMARY OF INVENTION

The invention involves the introduction of a compound that macerates the keratinous layer and second compound that disperses in the first to form a boundary between the infected cells and the healthy cells.

The first compound macerates the hyperkeratotic cells and creates an environment that the the film forming polymer will disperse into and encase the cells infected with Verrucae Vulgaris.

The film forming polymer will be generally miscible in the first compound that macerates the hyperkeratotic cells thus dispersing the polymer. Once the polymer is dispersed in the area infected by the wart virus the solvent that the polymer is dissolved in will evaporate. The encapsulated hyperkeratotic cells are now easily remove without pain through the natural shedding caused by the upward migration of new basale cells.

### BRIEF DESCRIPTION OF THE DRAWINGS

FIG. 1 is a schematic of a cross-sectional view of an area of skin with a wart showing a compound forming a boundary between the infected cells and healthy cells.

### DETAILED DESCRIPTION OF INVENTION

The invention involves the introduction of a compound in the keratinous spinosum layer. Most warts are characterized as having a hard and coarse center that is porous. The compound that is part of the invention penetrates the porous keratinous layer which is chiefly in the spinous layer. Once the compound has fully saturated the wart it will form a boundary between the infected epidermis and the healthy skin cells. The compound hardens and forms a solid mass.

The compound acts in two different ways. The first is that it binds very tightly to the infected keratinous cells and does not bind to the healthy epithelial cells. This difference in binding allows for the mechanical tearing of the infected cells from the healthy ones. The second mechanism is the upward migration of new basale cells will push the encased infected cells to the surface where the hardened mass will be removed.

The ability of the compound to penetrate into the infected cells and not interact with the healthy cells is one of the key aspects of the compound. To aid in the penetration of the compound the overburden of calloused skin should be removed as much as possible. Also, the saturation into the wart is aided by macerating the keratinous skin. The wart can be macerated with several compounds. Water and Acetic acid are examples of such compounds. The macerated skin saturated in water or acetic acid allows the compound to disperse in the wart due to the compound being somewhat miscible.

Repeated applications of the compound may be required to assure complete penetration of the compound throughout the infected tissue.

The compound only needs to form a boundary that separates the infected cells from the healthy cells. Examples of compounds that will achieve this effect are:
Nitrocellulose in solution
Polyphenyl methylsiloxane in solution
Acrylate terpolymer copolymer.

Other compounds can achieve the same results but the patent is not limited to the exact compound used; but the mechanism that various film forming polymers cause to happen. The compound that macerates the skin is also not unique but the combination of the two types of compounds makes the polymers effective.

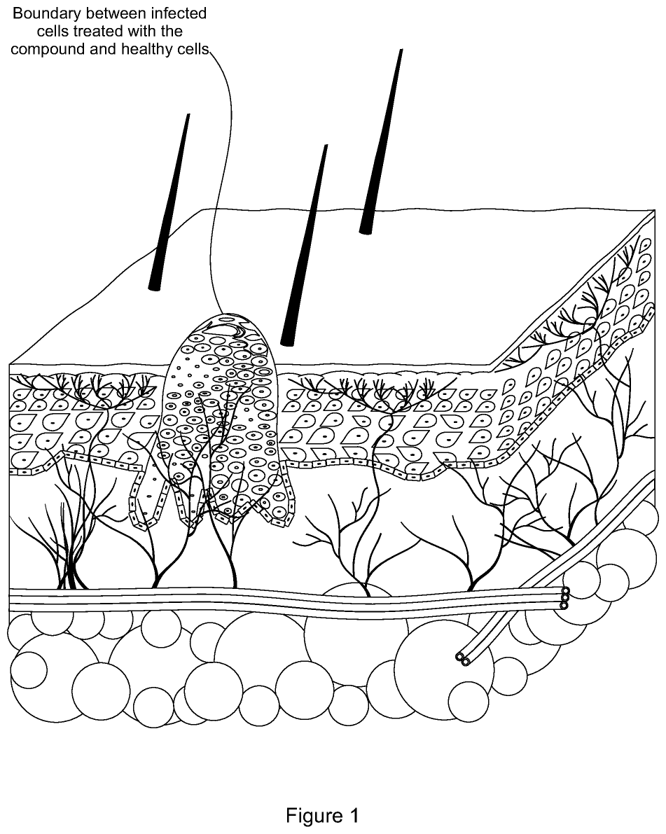

Other treatments rely on using a collodion to carry a keratolytic agent to localize the action of the keratolytic agent. The collodion is specifically intended to stay on the surface of the skin and not to penetrate into the wart infected skin. Refer to FIG. 1.

### Treatment Modalities

The following are three techniques for the introduction of the compound into the infected skin layer but does not represent all the possible techniques and is for illustration purposes only.

### Topical Application

This technique requires the soaking of the wart in water or an application of 4% Acetic acid for 5 to 15 minutes. The longer the wart is in contact with the water or acetic acid the better the keratinous skin is macerated. The polymer compound is then applied to the affected area and is allowed penetrate completely into the infected skin. For this modality to be the most effective it will require the removal of calloused skin around and over the wart. This can be accomplished with a scalpel or liquid nitrogen to cause the skin to blister.

After the application of the collodion, the volatile elements of the compound will have evaporated after 5-10 minutes leaving the only the hard aspect of the compound. Due to the porosity of the infected epidermal cells, the compound will only penetrate up to the point that it encounters healthy spinosum, basal or dermis cells.

Repeated applications of the compound may be required to assure complete penetration of the compound throughout the infected tissue.

Over the course of 2-6 weeks the natural upward migration of in the Stratum Basale will drive all the encapsulated cells to the surface and will eventually desquamate.

### Topical Application and Cryosurgery

This involves the same steps as the previous technique with the addition of Cryosurgery. After the compound has fully penetrated the infected skin cells the wart area is treated with liquid nitrogen. The liquid nitrogen causes the healthy skin to blister around the encapsulated infected skin.

The blistering will release the encapsulated infected tissue from the healthy tissue.

Intralesional Injection of Compound Directly into the Infected Tissue

This achieves the creation of a boundary between the infected cells and the healthy cells by placing the compound directly at the edges of the infected cells.

The placing of the compound at the edges of the infected cells is accomplished with a conventional syringe. The injection needle is inserted subdermally just underneath the wart from several locations around the wart, such that the solution is infiltrated directly below the wart, between the wart tissue and the surrounding skin tissue.

1. A method for treating a subject having a wart in an affected area of skin, the method comprising:
macerating the affected area with a first compound such that the affected area becomes saturated with the first compound;
applying a second compound to the saturated affected area such that the second compound disperses into the first compound to penetrate the skin of the affected area, the second compound comprising a film forming polymer and a solvent miscible with the first compound;
wherein, upon evaporation of the solvent, the film forming polymer hardens to form a boundary between healthy skin cells and infected skin cells within the affected area.

2. The method of claim 1, wherein the first compound comprises water or acetic acid.

3. The method of claim 2, wherein the macerating the affected area comprises soaking the affected area in the water or acetic acid for between about 5 and about 15 minutes.

4. The method of claim 1, wherein the film forming polymer of the second compound comprises nitrocellulose, polyphenyl methyl siloxane, or acrylate terpolymer copolymer.

5. The method of claim 1, wherein the second compound comprises collodion.

6. The method of claim 1, wherein applying the second compound comprises applying the second compound to the surface of the skin of the affected area.

7. The method of claim 1, wherein applying the second compound comprises injecting the second compound subdermally into the wart at an edge thereof.

8. The method of claim 1, further comprising re-applying the second compound to the affected area.

9. The method of claim 1, further comprising treating the affected area with liquid nitrogen after the second compound has penetrated the skin of the affected area.

10. The method of claim 1, further comprising mechanically tearing the infected skin cells from the healthy skin cells after the film forming polymer of the second compound has hardened.

11. The method of claim 1, further comprising removing an overburden of calloused skin in the affected area prior to macerating the affected area.

12. The method of claim 11, wherein the overburden is removed with a scalpel or with liquid nitrogen.
